(19) United States
(12) Patent Application Publication
(10) Pub. No.: US 2010/0302046 A1
(43) Pub. Date: Dec. 2, 2010

(54) STETHOSCOPE ADAPTED TO COMPRISE A RADIATION MONITOR METHOD AND DEVICE

(76) Inventor: MANUEL L. KARELL, Alameda, CA (US)

Correspondence Address: MANUEL L KARELL, 1084 JOST LN, ALAMEDA, CA, 94502, US

(21) Appl. No.: 12/781,790

(22) Filed: May 17, 2010

Related U.S. Application Data
(60) Provisional application No. 61/181,395, filed on May 27, 2009.

(51) Int. Cl. G08B 17/12; A61B 7/04

(52) U.S. Cl. 340/600; 381/67

(57) ABSTRACT

A method and device for monitoring a healthcare worker's exposure to radiation wherein a stethoscope is adapted to comprise a radiation monitor. The radiation monitor may be of instruments for measuring radiation including Geiger-Muller counters, scintillation detectors, photographic emulsions and various ionization chambers. The stethoscope may be acoustic or electronic or combined.

### CROSS REFERENCE TO RELATED PATENTS

This application pertains to PROVISIONAL APPLICATION NO. 61/181,395 filed on May 27, 2009.

### STATEMENT REGARDING FEDERAL SPONSORED RESEARCH AND DEVELOPMENT

NOT APPLICABLE

### NAMES OF PARTIES TO JOINT RESEARCH AGREEMENT

NOT APPLICABLE

### TECHNICAL FIELD

The present invention relates to medical examination instruments capable of providing several functions in a single instrument. In particular, the present invention relates generally to stethoscopes used for medical diagnostic purposes combined with radiation monitoring for monitoring energy radiations, such as electrons, X-rays, protons, photons, alpha particles, beta particles, neutrons and other emanations.

### BACKGROUND ART

Subatomic particles or electromagnetic waves that are energetic enough to detach electrons from atoms or molecules are called ionizing radiation. The occurrence of ionization depends on the energy of the impinging individual particles or waves, and not on their number. If particles or waves do not carry enough energy they are not ionizing. Examples of ionizing particles are energetic alpha particles, beta particles, and neutrons. The ability of an electromagnetic wave (photons) to ionize an atom or molecule depends on its frequency.

Ionizing radiation comes from radioactive materials, x-ray tubes, particle accelerators, and is present in the environment. It is invisible and not directly detectable by human senses, so instruments such as Geiger counters are usually required to detect its presence. It has many practical uses in medicine, research, construction, and other areas, but presents a health hazard if used improperly. Exposure to radiation at high doses causes damage to living tissue, resulting in skin burns, radiation sickness and death, and exposure at low doses result in malignant and/or non-malignant tumors and genetic damage.

Today, many people work in an environment that may become dangerous with increased levels of radioactivity. There is expanding utilization of radioactive materials in many scientific and industrial fields including medicine. There is growing concern that terrorists or others may at some time use radioactive or nuclear material in the construction of a nuclear weapon or “dirty bomb.” There is increasing need for health care providers to have with them improved equipment for detecting and monitoring radiation.

A physician, nurse, or other health care worker, normally keeps his/her stethoscope in his/her possession at all times; whereas, other instruments have a tendency to being mislaid and unavailable when needed.

The stethoscope enables listening to sounds for diagnostic purposes. The prior art of the stethoscope frequently shows the combination of other functions. For example, U.S. Pat. No. 5,989,186 discloses a stethoscope in which the chest piece comprises a light. U.S. Pat. No. 2,566,687 discloses a stethoscope in which the chest piece is combined with a light and tongue depressor. U.S. Pat. No. 6,510,918 is a combined stethoscope and reflex hammer. U.S. Pat. No. 4,672,975 discloses a stethoscope with an electronic device which forms the image of a beating heart on a display. U.S. Pat. No. 4,377,727 is of a stethoscope having a means for measuring pulse frequency.

Stethoscopes are acoustical or electronic and may be combined as in U.S. Pat. No. 5,774,563. A radiation monitor can be made to be very portable, as stick-on self-indicating instant radiation dosimeter in U.S. Pat. No. 7,227,158; and portable by combining with a time piece (watch) as in U.S. Pat. Nos. 4,608,655, 4,733,383, 7,351,985 and as in U.S. patent application Ser. No. 11/525,883.

The prior art also discloses the stethoscope having a transmission of data unit: examples are U.S. Pat. No. 5,550,902, U.S. Pat. No. 5,841,846, U.S. Pat. No. 6,533,736 and U.S. patent application Ser. No. 10/294,960.

Stethoscopes are used in hazardous material environments (HAZMAT) as in U.S. Pat. No. 7,182,733.

U.S. Patents referenced above are referenced in their entirety for contained within said patents are methods and procedures directly pertaining to the instant invention.

### DISCLOSURE OF THE INVENTION

Generally, the instant invention provides a method for monitoring a healthcare worker's exposure to radiation by having the worker carry a stethoscope comprising a radiation monitoring means, wherein the monitoring means alerts the worker of radiation exposure.

The instant invention is a stethoscope device capable of providing several functions in a single instrument, especially the combination of a stethoscope with a radiation monitor.

The instant invention generally involves the chest piece of a stethoscope. The chest piece is utilized for application to the chest or other part of a patient for listening to sounds. Operationally attached to or within the chest piece is a power supply for providing energy. Operationally attached to or within the chest piece is a radiation monitoring means for monitoring energy radiations, such as electrons, X-rays, protons, alpha particles, neutrons and/or other emanations. Radiation monitoring methods and devices are well known in prior art. They have been adapted to be inserted into consumer products such as pens and watches; the instant invention inserts them into a stethoscope.

A great variety of instruments for measuring radiation exist in the art, including Geiger-Muller counters, scintillation detectors, photographic emulsions and various ionization chambers. These are capable of detecting even very low levels of radiation. Radiation monitoring instruments are used both for area monitoring and for individual monitoring. The instruments used for measuring radiation levels are referred to as area survey meters (or area monitors) and the instruments used for recording the equivalent doses received by individuals working with radiation are referred to as personal dosimeters (or individual dosimeters).

Dosimeters measure an absolute dose received over a period of time. Ion-chamber dosimeters resemble pens, and can be clipped to one's clothing. Film-badge dosimeters enclose a piece of photographic film, which will become exposed as radiation passes through it. Ion-chamber dosimeters must be periodically recharged, and the result logged. Film-badge dosimeters must be developed as photographic emulsion so the exposures can be counted and logged; once developed, they are discarded. Another type of dosimeter is the thermoluminescent dosimeter (TLD). These dosimeters contain crystals that emit visible light when heated, in direct proportion to their total radiation exposure. Like ion-chamber dosimeters, TLDs can be re-used after they have been ‘read’. The prior art of radiation monitors have various indicate means to display or listen for radiation such as flashing LEDs or audio chirp or other means and may have remote operation. The stethoscope may be combined with any of these instruments.

In general, the stethoscope containing a radiation monitoring means will contain a means of power to provide energy of operation, an electronic circuit for monitoring radiation, a radiation detector that detects an intensity of at least one type of radiation, a radiation accumulator that receives and accumulates the detected intensity, a comparator that determines whether the accumulated intensity exceeds at least one threshold level, and an output device that is configured for indicating whether the accumulated intensity exceeds the threshold level.

The monitoring of radiation can be done by means of Geiger-Muller counter, which has been used in individual dosimeters. A monitor may be built on the basis of a microprocessor to combine radiation monitoring with timekeeping. There are smart radiological dosimeters designed to provide visual indication of accumulated dose with additional enhancements designed to give an extra level of assurance of monitoring and evaluating the radiological risks encountered. A stethoscope may comprise a Geiger-Muller counter in which the output is predetermined shapes.

A radiation monitoring means may have a pulse conversion means for converting the received pulses to an output to indicate integrated energy or it may comprise a radiation detector producing rate information related to real time radiation exposure rate.

### BRIEF DESCRIPTION OF THE DRAWINGS

FIG. 1 is a stethoscope in which the chest piece comprises a radiation monitor

FIG. 2 is a stethoscope in which the chest piece has a timekeeping unit and a radiation monitor

FIG. 3 is a stethoscope in which the chest piece has a timekeeping unit, a transmission unit and a radiation monitor

FIG. 4 is a schematic of an electronic circuit for monitoring radiation sending data to an indicator means

FIG. 5 is a schematic of a Geiger-Muller counter having an indicator with predetermined shapes

### DESCRIPTION OF THE PREFERRED EMBODIMENT

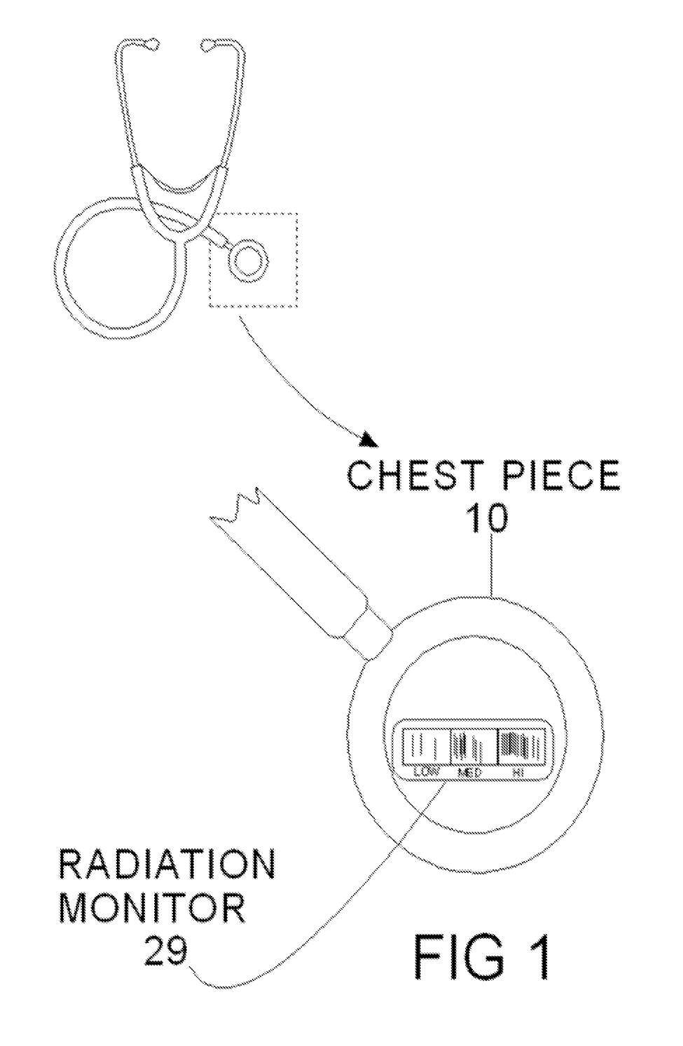

FIG. 1 is of a conventional stethoscope which includes a stethoscope head, flexible tubing, rigid conduits attached to flexible tubing, ear pieces attached to the conduits. The stethoscope usually includes a bell portion and diaphragm portion. A recessed area extends annularly on the stethoscope head between the bell and diaphragm portions. The chest piece is placed on the patient for making contact and listening to sounds or sending data. The instant invention contains a chest piece means 10 which includes a radiation monitoring means 29. In FIG. 1 the radiation monitoring is depicted as a digital read out as “LOW” “MED” “HI”; however, it should be understood that any form of radiation monitor indicator means can be utilized.

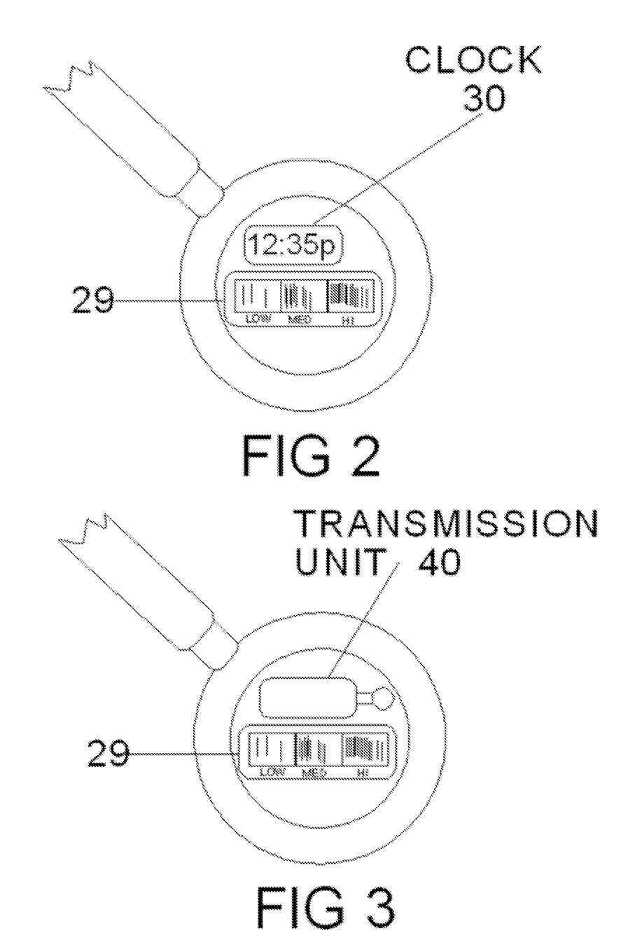

FIG. 2 shows a variant having a timekeeping unit means 30.

FIG. 3 shows a variant additionally having a data transmitting unit means 40 for sending data to a computer or other receiving unit.

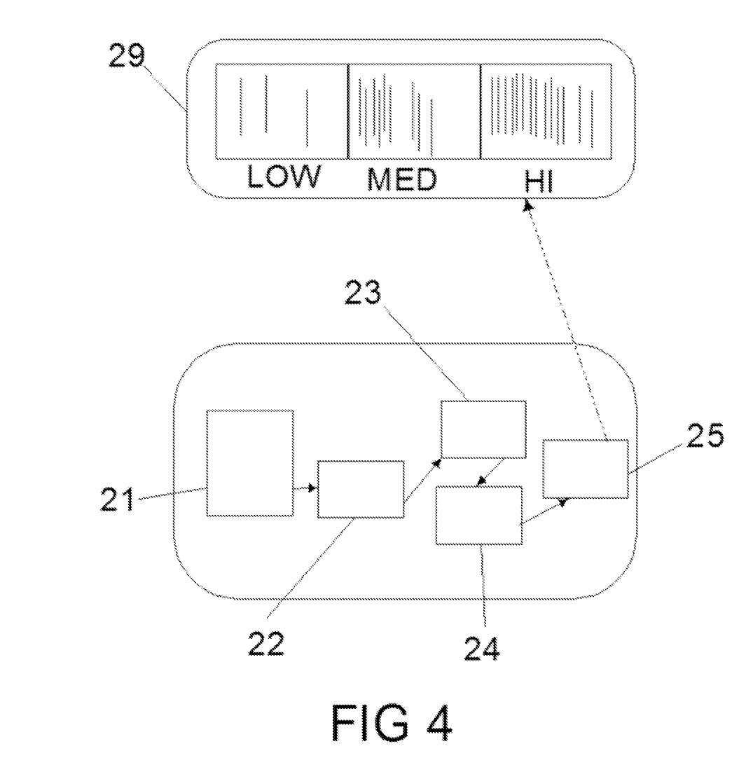

FIG. 4 is a schematic electronic circuit for a radiation monitoring means with digital readout 29, comprising an energy power means 21 to provide energy of operation, an electronic circuit means for monitoring radiation 22, a radiation detector means that detects an intensity of at least one type of radiation 23, a radiation accumulator means 24 that receives and accumulates the detected intensity from said radiation detector, a comparator means 25 that determines whether the accumulated intensity exceeds at least one threshold level, and an output device means 29 depicted in FIG. 4 as a digital readout.

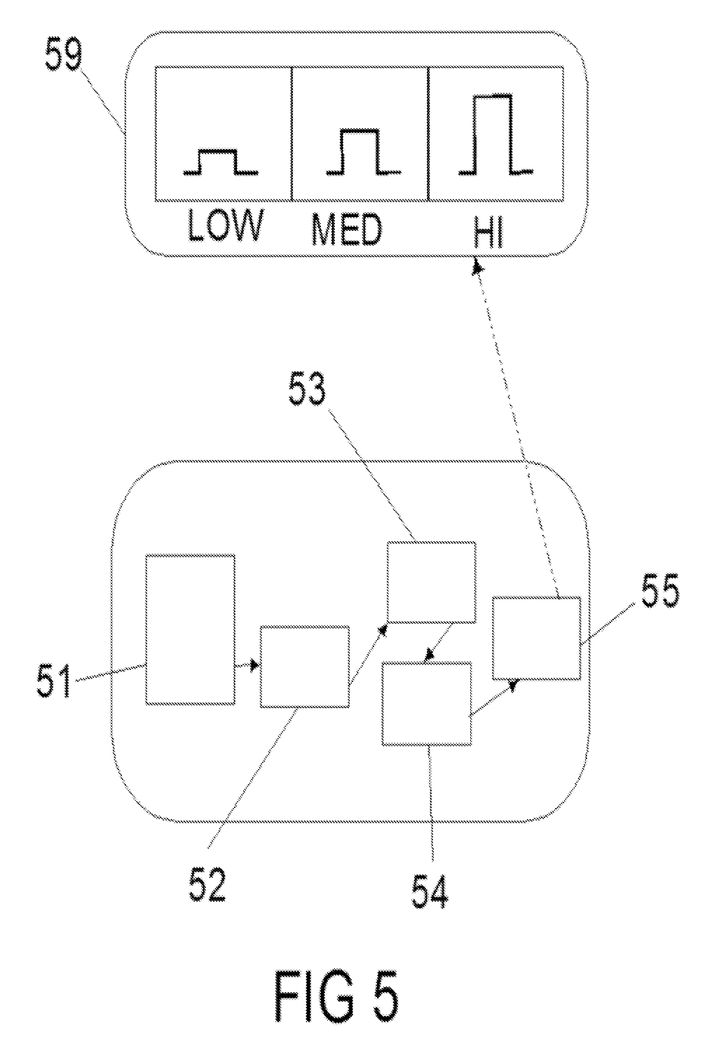

FIG. 5 is a schematic electronic circuit for a radiation monitoring means with shaped digital readout 59 having a power supply means 51 for providing energy of operation connected to a micro controller means 52 operatively connected to a Geiger-Muller counter means 53 for detecting radiation and for delivering corresponding signals to the micro controller means 52 for calculation of an effective radiation dose. A voltage pulse converter means 54 connected between the micro controller means 52 and the Geiger-Muller counter means 53 for transforming a low voltage from the micro controller 52 into a constant high voltage for transmission to the Geiger-Muller counter means 53. A pulse former means 55 connected to and between the Geiger-Muller counter and the micro controller for converting impulses from the Geiger-Muller counter into predetermined shapes for the indicator means 59 of the radiation monitoring means.

Prior art wherein the chest piece of the stethoscope contains a timekeeping unit is well known. Prior art of portable radiation monitors which can readily be inserted into the chest piece is well known.

While I have disclosed the preferred embodiments of my invention, it will be obvious to those skilled in the art that changes, variations and modifications may be made therein without departing from the invention in its broader aspects and, therefore, the aim in the appended claims is to cover such changes and modifications as fall within the scope and spirit of the invention.

What I claimed is:

1. A method for monitoring a healthcare worker's exposure to radiation such method composing the steps of having the worker carry a stethoscope comprising a radiation monitoring means, wherein said monitoring means alerts said worker of radiation exposure.

2. A stethoscope adapted for radiation monitoring comprising: an acoustical stethoscope means or an electronic stethoscope means or a combined acoustical and electronic stethoscope means for listening to sounds of a patient; wherein said stethoscope means contains a chest piece means for application to said patient; and wherein said chest piece means is operatively adapted to comprise a radiation monitoring means for detecting or monitoring radiation emanations.

3. A stethoscope of claim 2 wherein said chest piece means operatively contains within or is attached to a power supply means for providing energy for operation.

4. A stethoscope of claim 2 wherein said radiation monitoring means is operatively within said chest piece means.

5. A stethoscope of claim 2 wherein said radiation monitoring means is operatively attached to said chest piece means.

6. A stethoscope of claim 2 wherein said radiation monitoring means comprises a power means for providing energy of operation, an electronic circuit means for monitoring radiation, a radiation detector means for detecting an intensity of at least one type of radiation, a radiation accumulator means for receiving and accumulating the detected intensity from said radiation detector, a comparator means that determines whether the accumulated intensity exceeds at least one threshold level, and an output device means that is configured for indicating whether the accumulated intensity exceeds the threshold level.

7. A stethoscope of claim 2 wherein said a radiation monitor means comprises a power supply means for providing energy of operation; a micro controller means operatively connected to said power supply unit for receiving power therefrom; a Geiger-Muller counter for detecting radiation and for delivering corresponding signals to said micro controller for calculation of an effective radiation dose therein; a voltage pulse converter means connected between said micro controller means and said Geiger-Muller counter means for transforming a low voltage from said micro controller into a constant high voltage for transmission to said Geiger-Muller counter; and a pulse former means connected to and between said Geiger-Muller counter and said micro controller for converting impulses from said Geiger-Muller counter into predetermined shapes for processing in said micro controller.
